(19) United States
(12) Patent Application Publication
(10) Pub. No.: US 2006/0259135 A1
(43) Pub. Date: Nov. 16, 2006

(54) Apparatus and method for replacing a cardiac valve

(75) Inventors: Jose L. Navia, Shaker Heights, OH (US); Jose A. Navia, Buenos Aires (AR); Carlos Oberti, Westlake, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 11/408,756

(22) Filed: Apr. 20, 2006

Related U.S. Application Data
(60) Provisional application No. 60/673,056, filed on Apr. 20, 2005.

(51) Int. Cl. A61F 2/24

(52) U.S. Cl. 623/2.11; 623/2.18; 623/2.38

(57) ABSTRACT

An apparatus for replacing a cardiac valve having at least two native valve leaflets includes an expandable support member with oppositely disposed first and second ends and a main body portion extending between the ends. The first and second ends respectively include a plurality of upper and lower wing members respectively having first and second magnetic components. The wing members extend from the main body portion and are spaced circumferentially thereabout. Secured within the main body portion is a prosthetic valve having at least two valve leaflets. The second end further includes at least two strut members spaced apart from each other and attached to at least one commissural section of the prosthetic valve. The magnetic components are magnetically attracted to one another so that, when the apparatus is placed in the valve annulus, the wing members are pulled toward one another to secure the prosthetic valve in the annulus.

### RELATED APPLICATION

This application claims priority from U.S. provisional patent application Ser. No. 60/673,056, filed on Apr. 20, 2005, the subject matter of which is incorporated herein by reference.

### TECHNICAL FIELD

The present invention relates to an apparatus and method for replacing a cardiac valve, and is particularly directed to an apparatus and method for the correction of mitral valve and tricuspid valve disorders via a minimally invasive, percutaneous approach.

### BACKGROUND OF THE INVENTION

There are two atrioventricular (AV) valves in the heart; one on the left side of the heart and one on the right side of the heart. The left side AV valve is the mitral valve and the right side AV valve is the tricuspid valve. Both of these valves are subject damage and dysfunction that requires that the valve be repaired or replaced.

The mitral and tricuspid valves differ significantly in anatomy. While the annulus of the mitral valve is generally D-shaped, the annulus of the tricuspid valve is more circular. The effects of valvular dysfunction vary between the mitral valve and the tricuspid valve. Mitral valve regurgitation has more severe physiological consequences to the patient than does tricuspid valve regurgitation, a small amount of which is tolerable.

In mitral valve insufficiency, the valve leaflets do not fully close and a certain amount of blood leaks back into the left atrium when the left ventricle contracts. As a result, the heart has to work harder by pumping not only the regular volume of blood, but also the extra volume of blood that regurgitated back into the left atrium. The added workload creates an undue strain on the left ventricle. This strain can eventually wear out the heart and result in morbidity. Consequently, proper function of the mitral valve is critical to the pumping efficiency of the heart.

Mitral and tricuspid valve disease is traditionally treated by either surgical repair with an annuloplasty ring or surgical replacement with a valve prosthesis. Surgical valve replacement or repair, however, is often an exacting operation. The operation requires the use of a heart-lung machine for external circulation of the blood as the heart is stopped and then opened during the surgical intervention. Once the heart is opened, the artificial cardiac valves and/or annuloplasty rings are sewed in under direct vision.

Surgical repair of the AV valves exposes patients (i.e., elderly patients) to many risks. A percutaneous procedure that could be performed under local anesthesia in the cardiac catheterization lab, rather than in cardiac surgery, could therefore offer tremendous benefits to these patients. Consequently, an apparatus for replacing a diseased AV valve using a minimally invasive, percutaneous approach would be very helpful in providing additional opportunities to treat patients with valvular insufficiency and/or end stage heart failure.

### SUMMARY OF THE INVENTION

In one aspect of the present invention, an apparatus for replacing a cardiac valve includes an expandable support member having oppositely disposed first and second ends and a main body portion extending between the ends. The main body portion of the expandable support member has an annular shape for expanding into position in the annulus of the cardiac valve. The first end of the expandable support member includes a plurality of upper wing members that extend from the main body portion and are spaced circumferentially apart about the main body portion. Each of the upper wing members has a first magnetic component. The second end of the expandable support member includes a plurality of lower wing members that extend from the main body portion. Each of the lower wing members has a second magnetic component. The second end also includes at least two strut members that are spaced apart from each other. The apparatus further includes a prosthetic valve secured within the main body portion of the expandable support member. The prosthetic valve has at least two valve leaflets that are coaptable to permit unidirectional flow of blood. Each of the at least two valve leaflets are joined together at at least two commissural sections that are spaced apart from each other. Each of the at least two commissural sections is attached to a respective one of the strut members to prevent prolapse of the valve leaflets. The first and second magnetic components are magnetically attracted to one another so that, when the apparatus is placed in the annulus of the cardiac valve, the upper and lower wing members are pulled toward one another to secure the prosthetic valve in the annulus.

In another aspect of the present invention, at least a portion of the expandable support member is treated with at least one therapeutic agent for eluting into cardiac tissue or a cardiac chamber.

In yet another aspect of the present invention, a method is provided for replacing a cardiac valve having at least two native valve leaflets. One step of the method includes providing a prosthetic valve having an expandable support member with a main body portion. The prosthetic valve further includes a plurality of upper wing members that extend from a first end of the main body portion and include a first magnetic component attached to each upper wing member. A corresponding plurality of lower wing members extend from an opposite second end of the main body portion and include a second magnetic component attached to each lower wing member. The second end also includes at least two strut members that are spaced apart from each other. The prosthetic valve also has at least two valve leaflets that are coaptable to permit unidirectional flow of blood. The main body portion of the prosthetic valve is then placed within the annulus of the cardiac valve to be replaced and expanded into engagement with the annulus of the cardiac valve to secure the prosthetic valve in the annulus. Next, the upper and lower wing members are deployed from a radially collapsed condition into a radially extended condition whereby the first and second magnetic components are magnetically attracted and pull the upper and lower wing members toward each other, in turn securing the prosthetic valve in the annulus of the native cardiac valve.

In another aspect of the present invention, an apparatus for replacing a cardiac valve having at least two native valve leaflets includes an expandable support member having oppositely disposed first and second ends and a main body portion extending between the ends. The main body portion of the expandable support member has an annular shape for expanding into position in the annulus of the cardiac valve. The first end of the expandable support member includes a plurality of upper wing members that extend from the main body portion. The second end of the expandable support member includes a plurality of lower wing members that extend from the main body portion. The upper and lower wing members include means for magnetically attracting the upper and lower wing members toward each other to secure the apparatus in the annulus of the native cardiac valve. The second end also includes at least two strut members that are spaced apart from each other. Each of the at least two valve leaflets are joined together at at least two commissural sections that are spaced apart from each other. Each of the at least two commissural sections is attached to a respective one of the strut members to prevent prolapse of the valve leaflets. A prosthetic valve having at least two valve leaflets that are coaptable to permit unidirectional flow of blood is secured within the main body portion of the expandable support member.

In still another aspect of the present invention, an apparatus for replacing a cardiac valve having at least two native valve leaflets includes an expandable support member having oppositely disposed first and second ends and a main body portion extending between the ends. The main body portion has an annular shape for expanding into position in the annulus of the cardiac valve. The first and second ends of the expandable support member respectively include a plurality of upper and lower wing members that extend from the main body portion and are spaced circumferentially apart about the main body portion. Each of the upper and lower wing members includes at least one attachment mechanism. The second end of the expandable support member further includes at least two strut members that are spaced apart from each other. The main body portion further includes a first end portion and a second end portion. The first and second end portions respectively include first and second magnetic ring components which are magnetically attracted to one another so that, when the apparatus is placed in the annulus of the cardiac valve, the first and second end portions of the main body portion are pulled toward one another to secure the expandable support member in the annulus. The apparatus also includes a prosthetic valve secured within the main body portion of the expandable support member. The prosthetic valve has at least two valve leaflets that are coaptable to permit unidirectional flow of blood. Each of the at least two valve leaflets are joined together at at least two commissural sections that are spaced apart from each other. Each of the at least two commissural sections is attached to a respective one of the strut members to prevent prolapse of the valve leaflets.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is a schematic sectional view of an apparatus for replacing a diseased cardiac valve in accordance with the present invention and illustrating the apparatus being delivered to the diseased valve in a collapsed condition through a percutaneous procedure;

FIG. 2 is a perspective view of the apparatus of FIG. 1 in a radially extended condition;

FIG. 3 is a view similar to FIG. 1 illustrating the placement of the apparatus in the annulus of the cardiac valve in the extended condition;

FIG. 4 is a schematic sectional view taken along line 4-4 in FIG. 3;

FIG. 5 is a view similar to FIG. 2 illustrating an alternative construction of the apparatus;

FIG. 6 is a view similar to FIG. 5 illustrating another alternative construction of the apparatus;

FIG. 7 is a schematic bottom view taken along line 7-7 in FIG. 3 with parts omitted for clarity;

FIG. 8 is a schematic top view taken along line 8-8 in FIG. 3;

FIG. 9 is a plan view of the apparatus in FIG. 6 illustrating an alternative construction of the apparatus;

FIG. 10 is a view similar to FIG. 9 illustrating another alternative construction of the apparatus;

FIG. 11 is a cross-sectional view showing an alternative embodiment of the apparatus;

FIG. 12 is a view similar to FIG. 2 illustrating another alternative embodiment of the apparatus;

FIG. 13 is a cross-sectional view of the apparatus shown in FIG. 12;

FIG. 14 is a perspective view showing an alternative embodiment of the apparatus in FIG. 6 having artificial chordae;

FIG. 15 is a schematic top view similar to FIG. 10 illustrating an alternative embodiment of the apparatus;

FIG. 16 is a perspective view illustrating an alternative embodiment of the apparatus;

FIG. 17 is a cross-sectional view of the apparatus in FIG. 16 in a non-extended condition;

FIG. 18 is a cross-sectional view of the apparatus in FIG. 16 in an extended condition;

FIG. 19 is a perspective view illustrating another alternative embodiment of the apparatus; and

FIG. 20 is a perspective view illustrating yet another alternative embodiment of the apparatus.

### DETAILED DESCRIPTION

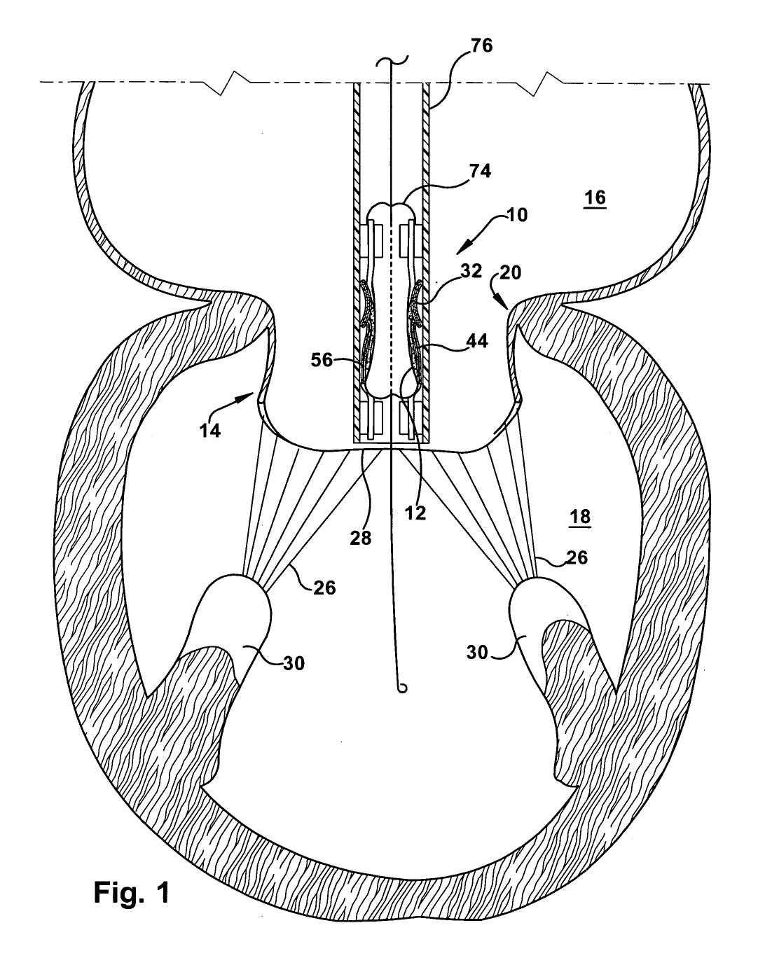

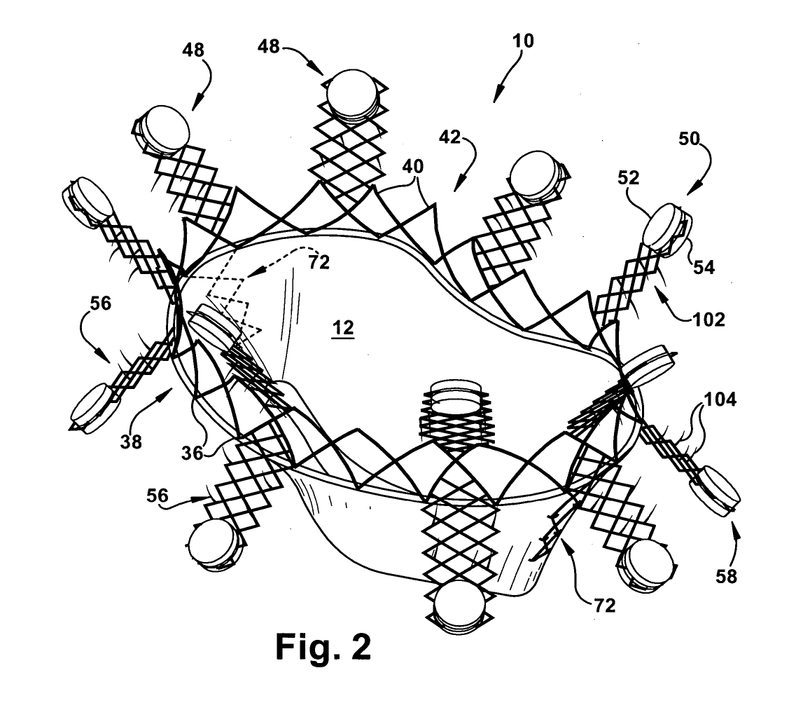

The present invention relates to an apparatus and method for replacing a cardiac valve, and is particularly directed to an apparatus and method for the correction of mitral valve and tricuspid valve disorders via a minimally invasive, percutaneous approach. As representative of the present invention, FIGS. 1 and 2 illustrate an apparatus 10 that includes a prosthetic valve 12 for replacing a dysfunctional cardiac valve, such as a mitral valve 14, by inserting the apparatus over the native mitral valve so that the prosthetic valve assumes the valvular function. It should be understood, however, that the apparatus 10 disclosed herein could also be used to replace other cardiac valves, such as a tricuspid, pulmonary, or aortic valve.

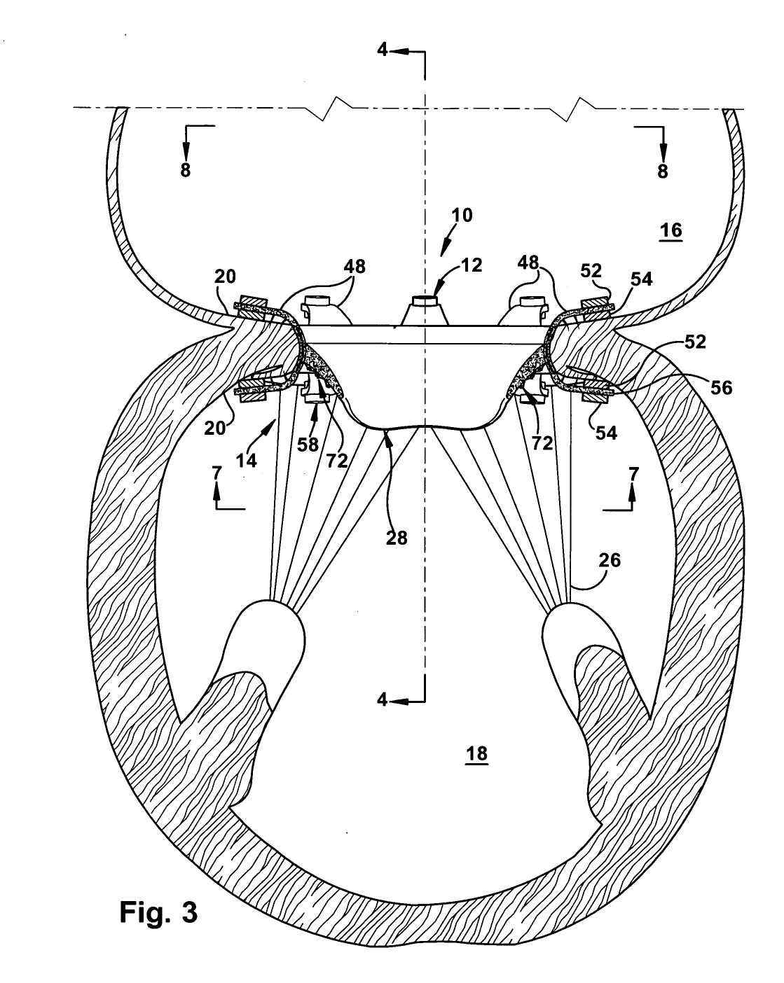

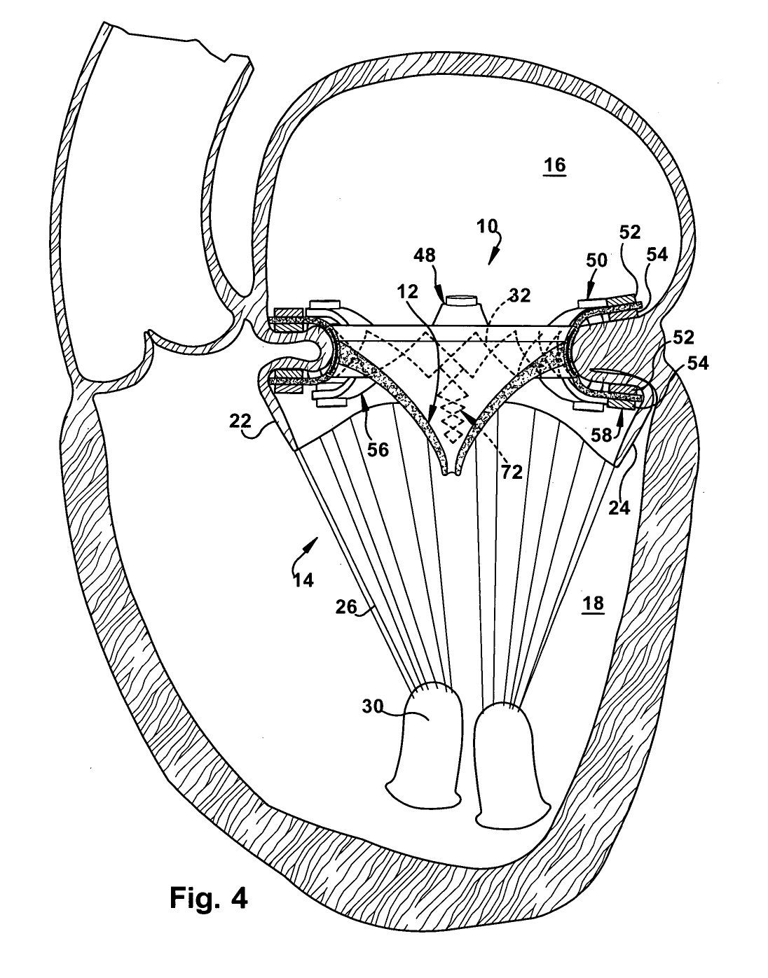

As shown in FIG. 1, the mitral valve 14 is located between the left atrium 16 and the left ventricle 18, and functions to prevent backflow of blood from the left ventricle into the left atrium during contraction. The mitral valve 14 has a D-shaped annulus 20 that defines the opening between the left atrium 16 and the left ventricle 18. The mitral valve 14 is formed by two leaflets; namely, the anterior leaflet 22 and the posterior leaflet 24 (FIG. 4). The anterior leaflet 22 extends along the generally planar base of the D-shaped valve annulus 20, while the posterior leaflet 24 extends arcuately around the curved portion of the D-shaped annulus of the mitral valve 14. Chordae tendinea 26 (FIG. 1) extend between the free edges 28 of both leaflets 22 and 24 and to the papillary muscles 30 in the left ventricle 16.

The apparatus 10 for replacing the dysfunctional mitral valve includes an expandable support member 32 (FIG. 2), commonly referred to as a stent, and a prosthetic valve 12. The expandable support member 32 has oppositely disposed first and second ends 42 and 38 and a main body portion 44 extending between the ends. The expandable support member 32 has a known stent configuration that allows it to be collapsed and expanded. The expandable support member 32 may be made from any suitable medical grade metal or plastic, including shape memory materials such as Nitinol, stainless steel, and/or titanium. Additionally, at least a portion of the apparatus 10 may be made from a bioabsorbable material including, for example, magnesium alloy, dendrimers, biopolymers such as thermoplastic starch, polyalctides, cellulose, and aliphatic aromatic copolyesters.

The expandable support member 32 comprises a continuous series of W-shaped segments 34 collectively forming a mesh-like configuration. It is contemplated, however, that other geometries may be used. The lower tips 36, as viewed in FIG. 2, of the W-shaped segments 34 form the second end 38 of the expandable support member 32, and the upper tips 40 of the W-shaped segments form the first end 42 of the expandable support member.

The expandable support member 32 is generally annular in shape. As shown in FIGS. 2-8, when the expandable support member 32 is expanded, the main body portion 44 has a concave cross-sectional shape. The flexible and expandable properties of the expandable support member 32 facilitate percutaneous delivery of the expandable support member, while also allowing the expandable support member to conform to the convex shape of the mitral valve annulus 20, for example.

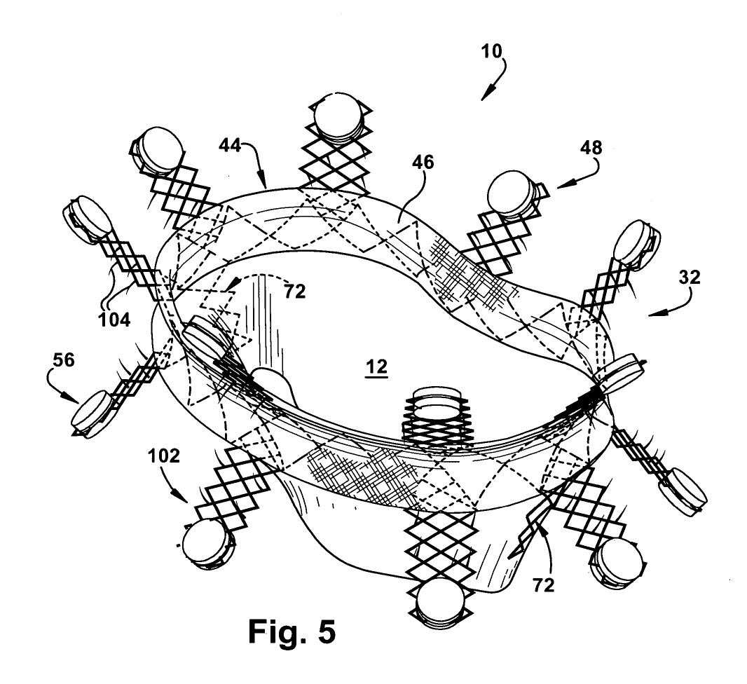

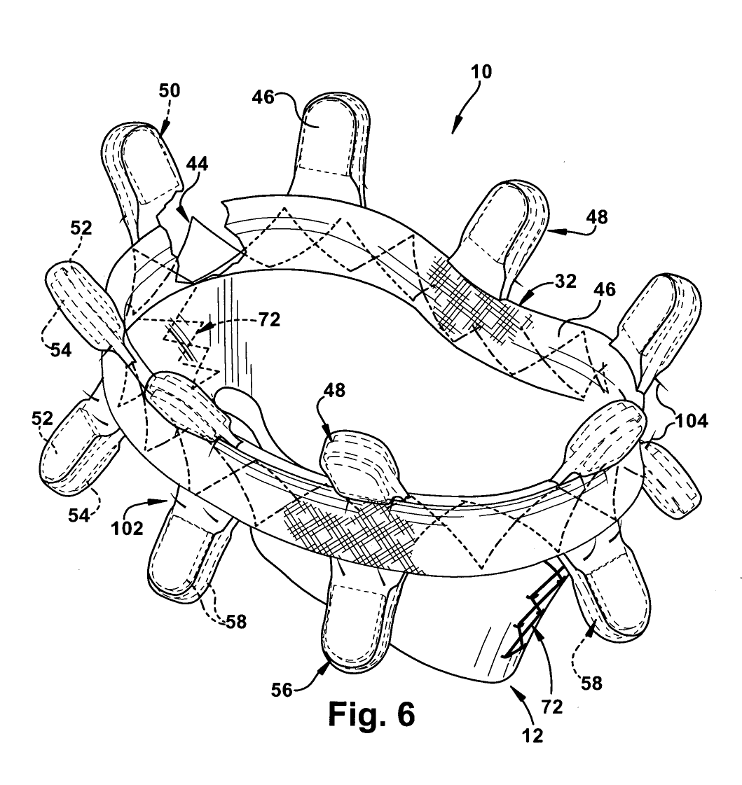

The apparatus 10 may further include a layer 46 of biocompatible material covering at least a portion of the expandable support member 32. The layer 46 of biocompatible material may be synthetic such as Dacron® (Invista, Wichita, Kans.), woven velour, polyurethane, polytetrafluoroethylene (PTFE), expanded PTFE, Gore-Tex® (W. L. Gore & Associates, Flagstaff, Ariz.), or heparin-coated fabric. Alternatively, the layer 46 may be a biological material such as bovine or equine pericardium, peritoneal tissue, an allograft, a homograft, a patient graft, or a cell-seeded tissue. The layer 46 can cover either the inside surface of the expandable support member 32, or the outside surface of the expandable support member, or can be wrapped around both the inside and outside surfaces. The layer 46 can cover either the inside surface of the expandable support member 32, the outside surface of the expandable support member, or can be wrapped around both the inside and outside surfaces. The layer 46 may be attached around the entire circumference of the expandable support member 32 or, alternatively, may be attached in pieces or interrupted sections to allow the expandable support member to more easily expand and contract. As shown in FIG. 5, for example, only the main body portion 44 of the expandable support member 32 may be covered with the layer 46 of biocompatible material. Alternatively, the entire apparatus 10 may be entirely covered with the layer 46 of biocompatible material (FIG. 6). By covering the first and second magnetic components 50 and 58 as shown in FIG. 6, the magnetic components are isolated from the blood and thereby improve the hemocompatibility of the apparatus 10.

The first end 42 of the expandable support member 32 comprises a plurality of upper wing members 48 that resemble arches and which extend integrally from the main body portion 44 generally in the proximal direction. In the embodiment illustrated in FIGS. 1-8, there are eight upper wing members 48 spaced about the circumference of the expandable support member 32. It should be understood, however, that more or less than eight upper wing members 48 could be used. The upper wing members 48 have a concave cross-sectional shape for conforming to the convex shape of the annulus of the cardiac valve, such as the mitral annulus 20. The upper wing members 48 are resiliently bendable and movable from the radially collapsed condition of FIG. 1 to the radially extended condition of FIGS. 2-8 for delivery and placement of the apparatus 10. As shown in FIGS. 2-8, each of the upper wing members 48 may include at least one attachment mechanism 102. The attachment mechanism 102 can include at least one barb 104, hook (not shown), or other similar means for embedding into a section of cardiac tissue to help secure the expandable support member 32 in the annulus of the cardiac valve.

As is also shown in FIGS. 2-8, each of the upper wing members 48 includes a first magnetic component 50. Each of the first magnetic components 50 comprise first and second magnetic members 52 and 54. The first and second magnetic members 52 and 54 are oppositely disposed on either side of the upper wing members 48. The first and second magnetic members 52 and 54 are comprised of material capable of producing a magnetic field. Examples of suitable materials include NdFeB (Neodymium Iron Boron), SmCo (Samarium Cobalt), and Alnico (Aluminum Nickel Cobalt). As shown in FIG. 2, the first and second magnetic members 52 and 54 may have a disc-like shape. It should be understood, however, that the first and second magnetic members 52 and 54 may have other shapes and sizes, such as the bullet-shaped wing members shown in FIG. 6, for example.

The first and second magnetic members 52 and 54 are secured to the upper wing members 48 as a result of the magnetic force between the first and second magnetic members. Alternatively, the first and second magnetic members 52 and 54 can be attached to the upper wing members 48 by gluing, suturing, pinning, clipping, or any other suitable attachment means. The amount of force exerted will depend on various factors, including the materials used and the size and number of first and second magnetic members 52 and 54. Different applications will call for different force ranges. For instance, application of the apparatus 10 to a patient's mitral valve 14 may call for a lesser or greater force as compared to application of the apparatus to a patient's tricuspid valve.

The second end 38 of the expandable support member 32 comprises a plurality of lower wing members 56 that resemble arches and which extend integrally from the main body portion 44 generally in the proximal direction. In the embodiment illustrated in FIGS. 1-8, there are eight lower wing members 56 spaced about the circumference of the expandable support member 32. It should be understood, however, that more or less than eight lower wing members 56 could be used. The quantity and circumferential location of lower wing members 56 correspond to the quantity and circumferential location of the upper wing members 48. Similar to the upper wing members 48, the lower wing members 56 also have a concave cross-sectional shape for conforming to the convex shape of the annulus of the cardiac valve, such as a mitral annulus 20. The lower wing members 56 are resiliently bendable and movable from the radially collapsed condition of FIG. 1 to the radially extended condition of FIGS. 2-8. As shown in FIGS. 2-8, each of the lower wing members 56 may include at least one attachment mechanism 102. The attachment mechanism 102 can include at least one barb 104, hook (not shown), or other similar means for engaging a portion of the native mitral valve leaflets 22 and 24, for example, to pin the leaflets back against the mitral valve annulus 20 (FIGS. 3 and 4).

As is also shown in FIGS. 2-8, each of the lower wing members 56 includes a second magnetic component 58. Each of the second magnetic components 58 comprises first and second magnetic members 52 and 54. The first and second magnetic members 52 and 54 are oppositely disposed on either side of the lower wing members 56. The first and second magnetic members 52 and 54 are comprised of material capable of producing a magnetic field. Examples of suitable materials include NdFeB (Neodymium Iron Boron), SmCo (Samarium Colbalt), and Alnico (Aluminum Nickel Cobalt).

The first and second magnetic members 52 and 54 are secured to the lower wing members 56 as a result of the magnetic force between the magnetic members. Alternatively, the first and second magnetic members 52 and 54 can be attached to the lower wing members 56 by gluing, suturing, pinning, clipping, or any other suitable means. The amount of force exerted will depend on various factors, including the materials used and the size and number of the first and second magnetic members 52 and 54. Different applications will call for different force ranges. For instance, application of the apparatus 10 to a patient's mitral valve 14 may call for a less or greater force as compared to application of the apparatus to a patient's tricuspid valve.

The prosthetic valve 12 of the present invention may comprise a stentless prosthetic valve. By “stentless” it is meant that the leaflets of the prosthetic valve 12 are not reinforced with a support structure, such as a stent or other similar structure. The prosthetic valve 12 is secured, for example, by sutures or other suitable means within the main body portion 44 of the expandable support member 32. Examples of prosthetic valves, such as the prosthetic valves disclosed in U.S. Pat. No. 5,156,621, which is hereby incorporated by reference in its entirety, are known in the art.

The prosthetic valve 12 may be fixed and preserved using a variety of known methods. The use of chemical processes for the fixation and preservation of biological tissues have been described and are readily available in the art. For example, glutaraldehyde, and other related aldehydes have seen widespread use in preparing cross-linked biological tissues. Glutaraldehyde is a five carbon aliphatic molecule with an aldehyde at each end of the chain, rendering it bifunctional. These aldehyde groups react under physiological conditions with primary amine groups on collagen molecules resulting in the cross-linking of collagen containing tissues. Methods for glutaraldehyde fixation of biological tissues have been extensively described and are well known in the art. In general, a tissue sample to be cross-linked is simply contacted with a glutaraldeyde solution for a duration effective to cause the desired degree of cross-linking within the biological tissue being treated.

Many variations and conditions have been applied to optimize glutaraldehyde fixation procedures. For example, lower concentrations have been found to be better in bulk tissue cross-linking compared to higher concentrations. It has been proposed that higher concentrations of glutaraldehyde may promote rapid surface cross-linking of the tissue, generating a barrier that impedes or prevents the further diffusion of glutaraldehdye into the tissue bulk. For most bioprosthesis applications, the tissue is treated with a relatively low concentration glutaraldehyde solution, e.g., typically between 0.1%-5%, for 24 hours or more to ensure optimum fixation. Various other combinations of glutaraldehyde concentrations and treatment times will also be suitable depending on the objectives for a given application. Examples of such other combinations include, but are not limited to, U.S. Pat. Nos. 6,547,827, 6,561,970, and 6,878,168, all of which are hereby incorporated by reference in their entirety.

In addition to bifunctional aldehydes, many other chemical fixation procedures have been described. For example, some such methods have employed polyethers, polyepoxy compounds, diisocyanates, and azides. These and other approaches available to the skilled individual in the art for treating biological tissues are suitable for cross-linking vascular graft tissue according to the present invention.

The prosthetic valve 12 may also be treated and preserved with a dry tissue valve procedure as described in U.S. Pat. No. 6,534,004, the entire contents of which are hereby incorporated by reference. Furthermore, the prosthetic valve 12 may be treated with anti-calcification solutions, such as XenoLogiX® treatment (Edwards Lifesciences, Irvine, Calif.) or the SynerGraf® (CryoLife, Inc., Kennesaw, Ga.) treatment process, and/or anti-calcification agents, such as alfa-amino oleic acid.

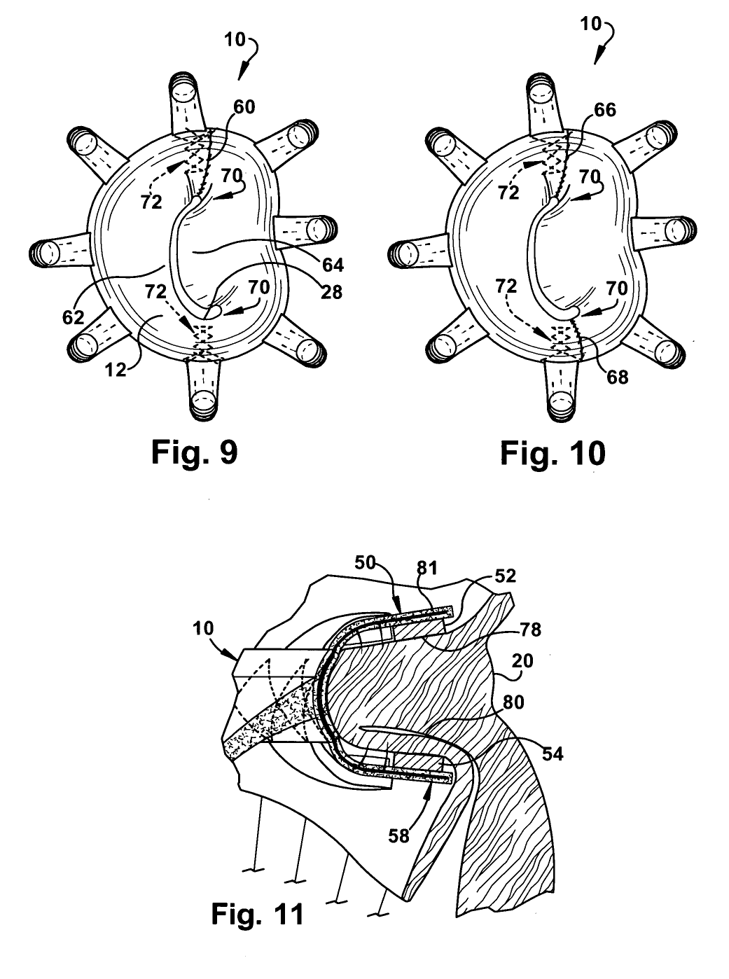

The prosthetic valve 12 can be made with only one piece of pericardial tissue, for example, as shown in FIG. 9. Where a single piece of pericardial tissue is used, a seam 60 is formed by suturing the ends of the tissue. Alternatively, the prosthetic valve 12 can be made with two pieces of pericardial tissue, one of which will form the first leaflet 62 and the other forms the second leaflet 64 of the prosthetic valve, as may be seen in FIG. 10. Where two pieces of pericardial tissue are used (FIG. 10), it is necessary to suture the tissue in two locations, thereby forming two seams 66 and 68. The seams 60, 66, and 68 are always placed at what will be the commissural sections 70 of the prosthetic valve 12, where the first leaflet 62 meets the second leaflet 64.

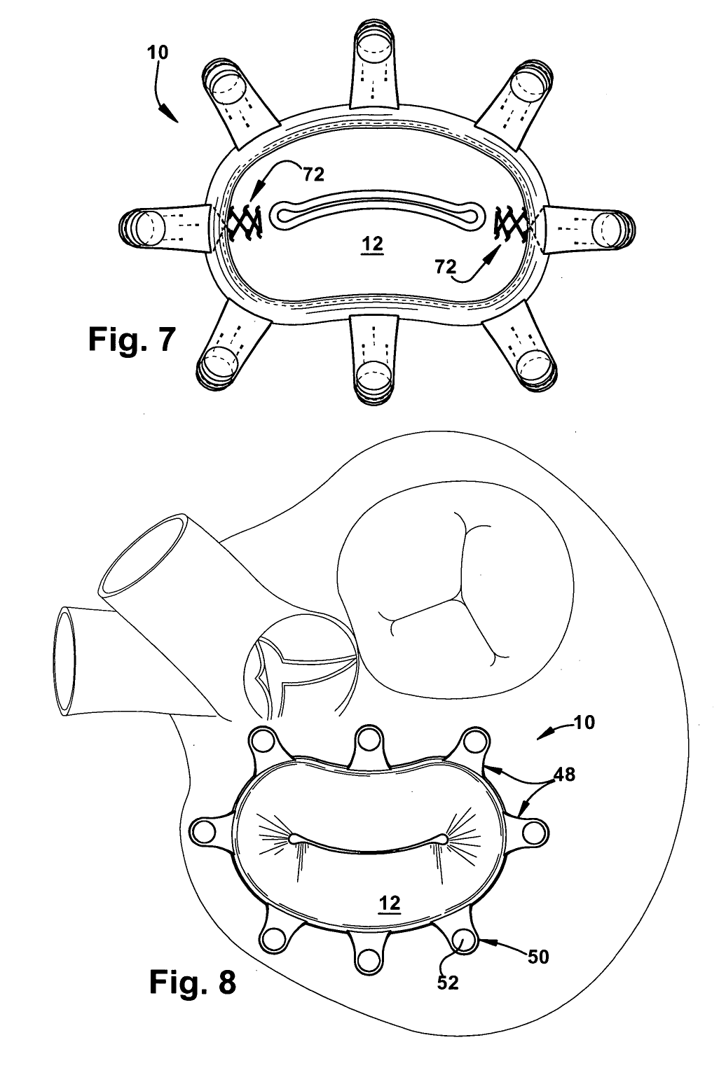

The second end 38 of the expandable support member 32 additionally includes at least two strut members 72. As shown in FIG. 7, the valve leaflets of the prosthetic valve 12 are joined together at at least two commissural sections 70 that are spaced apart from each other. Each of the at least two commissural sections 70 are attached to a representative one of the strut members 72 to prevent prolapse of the valve leaflets. The strut members 72 are securely attached to, and extend in a generally axial manner from, the expandable support member 32. The strut members 72 are securely connected to the prosthetic valve 12 by sutures (not shown), for example, and may be made from any suitable medical grade metal or plastic, including shape memory materials such as Nitinol, stainless steel, and/or titanium. As illustrated in FIG. 7, the strut members 72 have a bare metal configuration and do not extend beyond the length of the prosthetic valve 12. It is contemplated, however, that the configuration of the strut members 72 may be varied as needed. For example, the strut members 72 may be covered by a layer 46 of biocompatible material and extend beyond the length of the prosthetic valve 12.

At least a portion of the expandable support member 32 (FIG. 2) is treated with at least one therapeutic agent for eluting into cardiac tissue or a cardiac chamber. The therapeutic agent is capable of preventing a variety of pathological conditions including, but not limited to, arrhythmias, thrombosis, stenosis and inflammation. Accordingly, the therapeutic agent may include at least one of an anti-arrhythmic agent, anticoagulant, an antioxidant, a fibrinolytic, a steroid, an anti-apoptotic agent, and/or an anti-inflammatory agent. Optionally or additionally, the therapeutic agent may be capable of treating or preventing other disease or disease processes such as microbial infections and heart failure. In these instances, the therapeutic agent may include an inotropic agent, a chronotropic agent, an anti-microbial agent, and/or a biological agent such as a cell or protein. More specific types of these therapeutic agents are listed below, including other types of therapeutic agents not discussed above.

A plurality of portions of the expandable support member 32 may be separately treated with a different one of the therapeutic agents. For example, the main body portion 44 may be treated with an anti-inflammatory agent while each of the wing members 48 and 56 is separately treated with an anti-coagulant. Alternatively, the upper and lower wing members 48 and 56 may be separately treated with a different therapeutic agent. By treating the expandable support member 32 with different therapeutic agents, different medical conditions can be simultaneously treated. It should be appreciated that the expandable support member 32 may be treated with any combination and/or variation of the therapeutic agents mentioned above and discussed further below.

Examples of acceptable therapeutic agents include heparin, synthetic heparin analogues (e.g., fondaparinux), G(GP) II_{b}/IIIₐ inhibitors, vitronectin receptor antagonists, hirudin, antithrombin III, drotrecogin alpha; fibrinolytics such as alteplase, plasmin, lysokinase, factor XIIa, factor VIIa, prourokinase, urokinase, streptokinase; thrombocyte aggregation inhibitors such as ticlopidine, clopidogrel, abciximab, dextrans; corticosteroids such as aldlometasones, estradiols, such as 17β-estradiol, amcinonides, augmented betamethasones, beclomethasones, betamethasones, budesonides, cortisones, clobetasol, clocortolones, desonides, desoximetasones, dexamethasones, flucinolones, fluocinonides, flurandrenolides, flunisolides, fluticasones, halcinonides, halobetasol, hydrocortisones, methylpred nisolones, mometasones, prednicarbates, prednisones, prednisolones, triamcinolones; fibrinolytic agents such as tissue plasminogen activator, streptokinase, dipyridamole, ticlopidine, clopidine, and abciximab; non-steroidal anti-inflammatory drugs such as salicyclic acid and salicyclic acid derivatives, para-aminophenol derivatives, indole and indene acetic acids (e.g., etodolac, indomethacin, and sulindac), heteroaryl acetic acids (e.g., ketorolac, diclofenac, and tolmetin), arylpropionic acids (e.g., ibuprofen and derivatives thereof), anthranilic acids (e.g., meclofenamates and mefenamic acid), enolic acids (e.g., piroxicam, tenoxicam, phenylbutazone, and oxyphenthatrazone), gold compounds (e.g., auranofin, aurothioglucose, and gold sodium thiomalate), diflunisal, meloxicam, nabumetones, naproxen, oxaprozin, salsalate, celecoxib, rofecoxib; cytostatics such as alkaloids and podophyllum toxins such as vinblastin, vincristin; alkylants such as nitrosoureas and nitrogen lost analogues; cytotoxic antibiotics such as daunorubicin, doxorubicin, and other anthracyclins and related substances, bleomycin, and mitomycin; antimetabolites such as folic acid analogues, purine analogues and related inhibitors (e.g., mercaptopurine, thioguanine, pentostatin, and 2-chlorodeoxyadenosine), pyrimidine analogues (e.g., fluorouracil, floxuridine, and cytarabine), and platinum coordination complexes (e.g., cisplatinum, carboplatinum and oxaliplatinum); tacrolimus, azathioprine, cyclosporine, paclitaxel, docetaxel, sirolimus; amsacrin, irinotecan, imatinib, topotecan, interferon-alpha 2a, interferon-alpha 2b, hydroxycarbamide, miltefosin, pentostatin, porfimer, aldesleukin, bexarotene, and tretinoin; antiandrogens and antiestrogens; antiarrythmics, in particular antiarrhythmics of class I such as antiarrhythmics of the quinidine type (e.g., quinidine, dysopyramide, ajmaline, prajmalium bitartrate, and detajmium bitartrate); antiarrhythmics of the lidocaine type, (e.g., lidocaine, mexiletin, phenyloin, and tocainid); antiarrhythmics of class I C (e.g., propafenone, flecainide (acetate)); antiarrhythmics of class II, including betareceptor blockers such as metoprolol, esmolol, propranolol, metoprolol, atenolol, and oxprenolol; antiarrhythmics of class III such as amiodarone and sotalol; anti arrhythmics of class IV such as diltiazem, and verapamil; and other antiarrhythmics such as adenosine, orciprenaline, TC-912, and ipratropium bromide.

Other types of therapeutic agents may include digitalis glycosides such as acetyl digoxin/methyldigoxin, digitoxin, and digoxin; heart glycosides such as ouabain and proscillaridin; antihypertensives such as centrally effective antiadrenergic substances (e.g., methyldopa and imidazoline receptor agonists); calcium channel blockers of the dihydropyridine type, such as nifedipine and nitrendipine; ACE inhibitors (e.g., quinaprilate, cilazapril, moexipril, trandolapril, spirapril, imidapril, and trandolapril); angiotensin-II-antagonists (e.g., candesartancilexetil, valsartan, telmisartan, olmesartan medoxomil, and eprosartan); peripherally effective alpha-receptor blockers such as prazosin, urapidil, doxazosin, bunazosin, terazosin, and indoramin; vasodilators such as dihydralazine, diisopropyl amine dichloroacetate, minoxidil, and nitropiusside-sodium; other antihypertonics such as indapamide, codergocrin mesilate, dihydroergotoxin methane sulphonate, cicletanin, bosentan, and fluorocortisone; phosphodiesterase inhibitors, such as milrinone and enoximone, as well as antihypotonics (e.g., adrenergics and dopaminergic substances such as dobutamine, epinephrine, etilefrine, norfenefrine, norepinephrine, oxilofrine, dopamine, midodrine, pholedrine, and amezinium methyl) and partial adrenoreceptor agonists (e.g., dihydroergotamine); fibronectin, polylysines and ethylene vinyl acetates; and adhesive substances such as cyanoacrylates, beryllium, and silica.

Additional therapeutic agents may also include antibiotics and antiinfectives such as -lactam antibiotics (e.g., -lactamase-sensitive penicillins, including benzyl penicillins (penicillin G) and phenoxymethylpenicillin (penicillin V)); -lactamase-resistant penicillins, such as aminopenicillins, which include amoxicillin, ampicillin, and bacampicillin; acylaminopenicillins such as meziocillin and piperacillin; carboxypenicillines and cephalosporins (e.g., cefazolin, cefuroxim, cefoxitin, cefotiam, cefaclor, cefadroxil, cefalexin, loracarbef, cefixim, cefuroximaxetil, ceftibuten, cefpodoximproxetil, and cefpodoximproxetil); aztreonam, ertapenem, and meropenem; -lactamase inhibitors such as sulbactam and sulfamicillintosilates; tetracyclines such as doxycycline, minocycline, tetracycline, chlorotetracyc ine, oxytetracyc ine; aminoglycosides such as gentamicin, neomycin, streptomycin, tobramycin, amikasin, netilmicin, paromomycin, framycetin, and spectinomycin; makrolide antibiotics such as azithromycin, clarithromycin, erythromycin, roxithromycin, spiramycin, and josamycin; lincosamides such as clindamycin and lincomycin; gyrase inhibitors, such as fluoroquinolones, which include ciprofloxacin, ofloxacin, moxifloxacin, norfloxacin, gatifloxacin, enoxacin, fleroxacin, and levofloxacin; quinolones such as pipemidic acid; sulphonamides such as trimethoprim, sulphadiazin, and sulphalene; glycopeptide antibiotics such as vancomycin and teicoplanin; polypeptide antibiotics, such as polymyxins, which include colistin, polymyxin-b, and nitroimidazol derivatives (e.g., metronidazol and timidazol); aminoquinolones such as chloroquin, mefloquin, and hydroxychloroquin; biguanides such as proguanil; quinine alkaloids and diaminopyrimidines such as pyrimethamine; amphenicols such as chloramphenicol; rifabutin, dapsone, fusidinic acid, fosfomycin, nifuratel, telithromycin, fusafungin, fosfomycin, pentamidindiisethionate, rifampicin, taurolidine, atovaquone, and linezolid; virostatics such as aciclovir, ganciclovir, famciclovir, foscamet, inosine (dimepranol-4-acetamidobenzoate), valganciclovir, valaciclovir, cidofovir, and brivudin; tyrosine kinase inhibitors; anti-apoptotic agents such as caspase inhibitors (e.g., fluoromethylketone peptide derivatives), calpain inhibitors, cathepsin inhibitors, nitric oxide synthase inhibitors, flavonoids, vitamin A, vitamin C, vitamin E, vitamin D, pycnogenol, super oxidedismutase, N-acetyl cysteine, selenium, catechins, alpha lipoic acid, melatonin, glutathione, zinc chelators, calcium chelators, and L-arginine; Coumadin; beta-blockers; diuretics; spirolactone; TC-313; and natural products such as vinca alkaloids (e.g., vinblastine, vincristine and vinorelbine).

As noted above, the therapeutic agent may also include a biological agent. The biological agent may include organic substances such as peptides, proteins, enzymes, carbohydrates (e.g., monosaccharides, oligosaccharides and polysacchardies), lipids, phospholipids, steroids, lipoproteins, glycoproteins, glycolipids, proteoglycans, polynucleotides (e.g., DNA and RNA), antisense polynucleotides (e.g., c-myc antisense), antibodies (e.g., monoclonal or polycolonal) and/or antibody fragments (e.g., anti-CD34 antibody), bioabsorbable polymers (e.g., polylactonic acid), chitosan, extracellular matrix modulators, such as matrix metalloproteinases (MMP), which include MMP-2, MMP-9 and Batimastat; and protease inhibitors.

Biological agents may include, for example, agents capable of stimulating angiogenesis in the myocardium. Such agents may include vascular endothelial growth factor (VEGF), basic fibroblast growth factor (bFGF), non-viral DNA, viral DNA, and endothelial growth factors (e.g., FGF-1, FGF-2, VEGF, TGF). Other growth factors may include erythropoietin and/or various hormones such as corticotropins, gonadotropins, sonlatropin, thyrotrophin, desmopressin, terlipressin, oxytocin, cetrorelix, corticorelin, leuprorelin, triptorelin, gonadorelin, ganirelix, buserelin, nafarelin, and goserelin. Additional growth factors may also include cytokines, epidermal growth factors (EGF), platelet derived growth factor (PDGF), transforming growth factors- (TGF-), transforming growth factor- (TGF-), insulin-like growth factor-I (IGF-I), insulin-like growth factor-II (IGF-II), interleukin-1 (IL-1), interleukin-2 (IL-2), interleukin-6 (IL-6), interleukin-8 (IL-8), tumour necrosis factor- (TNF-), tumour necrosis factor- (TNF-), interferon- (INF-), colony stimulating factors (CSFs); monocyte chemotactic protein, and fibroblast stimulating factor 1.

Still other biological agents may include regulatory peptides such as somatostatin and octreotide; bisphosphonates (e.g., risedronates, pamidronates, ibandronates, zoledronic acid, clodronic acid, etidronic acid, alendronic acid, and tiludronic acid); fluorides such as disodium fluorophosphate and sodium fluoride; calcitonin and dihydrotachystyrene; histamine; fibrin or fibrinogen; endothelin-1; angiotensin II; collagens; bromocriptin; methylsergide; methotrexate; carbontetrachloride and thioacetamide.

The present invention may also be treated (i.e., seeded) with other biological agents, such as cells. Suitable cells may include any one or combination of eukaryotic cells. Additionally or optionally, the cells may be capable of producing therapeutic agents and/or genetically engineered to produce therapeutic agents. Suitable cells for use in the present invention include, for example, progenitor cells such as adult stem cells, embryonic stem cells, and umbilical cord blood stem cells. The cells may be autologous or allogenic, genetically engineered or non-engineered, and may include, for example, mesenchymal or mesodermal cells, including, but not limited to, endothelial progenitor cells, endothelial cells, and fibroblasts. Mixtures of such cells can also be used.

A variety of ex vivo or in vivo methods can be used to deliver a nucleic acid molecule or molecules, such as a gene or genes, to the cells. For example, the cells can be modified (i.e., genetically engineered) to produce or secrete any one or combination of the above therapeutic agents, including, but not limited to, anticoagulant agents, antiplatelet agents, antifibrinolytic agents, angiogenesis factors, and the like. Ex vivo gene transfer is a process by which cells are removed from the body using well known techniques, genetically manipulated, usually through transduction or transfection of a nucleic acid molecule into the cells in vitro, and the returned to the body for therapeutic purposes. This contrasts with in vivo genetic engineering where a gene transfer vector is administered to a patient resulting in genetic transfer into cells and tissues in the intact patient. Ex vivo and in vivo gene transfer techniques are well known to one of skill in the art.

To treat the present invention with at least one therapeutic agent, a variety of methods, agents, and compositions may be used. For example, the therapeutic agent can be simply linked to the stent surface, embedded and released from within polymer materials, such as a polymer matrix, or surrounded by and released through a carrier. Several approaches to treating medical devices with therapeutic agents exist. Some therapeutic agents can be loaded directly onto metallic surfaces; however, a coating composition, typically comprised of at least one polymer and at least one therapeutic agent, is usually used to treat drug-eluting devices. The coating composition ensures retention of the therapeutic agent during deployment and modulates elution kinetics of the therapeutic agent. By altering the release kinetics of different therapeutic agents in the same coating composition, distinct phases of a given disease process may be targeted.

The present invention may be treated with a coating composition comprising at least one therapeutic agent and at least one dendrimer, polymer or oligomer material. The dendrimer(s), polymer(s) and/or oligomer(s) may be of various types and from various sources, including natural or synthetic polymers, which are biocompatible, bioabsorbable, and useful for controlled release of the therapeutic agent. For example, synthetic polymers can include polyesters, such as polylactic acid, polyglycolic acid, and/or combinations thereof, polyanhydrides, polycaprolactones, polyhydroxybutyrate valerates, and other biodegradable polymers or mixtures of copolymers thereof. Natural polymeric materials can include proteins such as collagen, fibrin, elastin, extracellular matrix components, other biologic agents, and/or mixtures thereof.

The polymer material or mixture thereof of the coating composition can be applied with the therapeutic agent on the surface of the present invention and can comprise a single layer. Optionally, multiple layers of the polymer material can be applied to form the coating composition. Multiple layers of the polymer material can also be applied between layers of the therapeutic agent. For example, the polymeric layers may be applied sequentially, with the first layer directly in contact with the uncoated surface of the apparatus and a second layer comprising the therapeutic agent and having one surface in contact with the first layer and the opposite surface in contact with a third layer of polymeric material which is in contact with the surrounding tissue. Additional layers of the polymeric material and therapeutic agent can be added as required.

Alternatively, the coating composition can be applied as multiple layers comprising one or more therapeutic agents surrounded by polymer material. For instance, the coating composition can comprise multiple layers of a single therapeutic agent, one or more therapeutic agents in each layer, and/or differing therapeutic agents in alternating layers. Alternatively, the layers comprising the therapeutic agent can be separated from one another by a layer of polymer material.

The coating composition may further comprise at least one pharmaceutically acceptable polymers and/or pharmaceutically acceptable carriers, for example, non-absorbable polymers, such as ethylene vinyl acetate and methylmethacrylate. The non-absorbable polymer, for example, can aid in further controlling release of the therapeutic agent by increasing the molecular weight of the coating composition and thereby delaying or slowing the rate of release of the therapeutic agent.

The coating composition can be applied to the present invention using standard techniques to cover the entire surface of the apparatus, or partially, as a single layer in a dot matrix pattern, for example. The coating composition can be applied using various techniques available in the art, such as dipping, spraying, vapor deposition, an injection-like and/or a dot matrix-like approach. Upon contact of the coating composition with adjacent tissue where implanted, the coating composition can begin to degrade in a controlled manner. As the coating composition degrades, the therapeutic agent is slowly released into adjacent tissue and the therapeutic agent is eluted so that the therapeutic agent can have its effect locally.

Where the therapeutic agent comprises a biological agent, such as cells, the biological agent can be coated directly onto the surface of the present invention or, alternatively, they can be incorporated into the polymeric material (e.g., into a polymer matrix). Such biological agents may also be included within at least one microscopic containment vehicle (e.g., a liposome, nanocapsule, nanoparticle, micelle., synthetic phospholipid, gas-dispersion, emulsion, microemulsion, nanosphere, and the like) that can be stimulated to release the biological agent(s) and/or that release the biological agent(s) in a controlled manner. The microscopic containment vehicle can be coated onto the surface of the present invention or incorporated into the polymeric material. Where the biological agent comprises cells, for example, the cells can be induced to produce, activate, and/or release their cellular products (including one or more therapeutic agents) by an external stimulation device (e.g., an electrical impulse). Alternatively, cells can constitutively release one or more therapeutic agents at a desired level.

To enable delivery and deployment of the apparatus 10 in the mitral valve 14, for example, the apparatus is positioned about a balloon 74 (FIG. 1) for expanding the main body portion 44 of the expandable support member 32 into full and complete contact with the annulus 20 of the mitral valve. The balloon 74 may have an hourglass shape to conform to the concave cross-sectional configuration of the main body portion 44. In addition, releasable constraining wires (not shown) are used to temporarily hold the upper wing members 48 and the lower wing members 56 in the radially collapsed conditions shown in FIG. 1 during delivery and placement of the apparatus 10. The constraining wires can be made from a variety of different materials including metals, polymers, synthetics, fabrics, and biological tissues. With the upper wing members 48, the lower wing members 56, and the main body portion 44 of the expandable support member 32 in their collapsed conditions, the apparatus 10 is then loaded into the end of a 16 to 22 French catheter 76 in a known manner.

To replace the mitral valve 14 with the apparatus 10 using a percutaneous (or intravascular) approach, the apparatus is first sized for the particular mitral valve using fluoroscopic and/or echocardiographic data. The catheter 76 is then introduced into either the right or left jugular vein (not shown), a femoral vein (not shown), or the subclavian vein (not shown) using a known percutaneous technique, such as the Seldinger technique, and is advanced through the superior or inferior vena cava (not shown) to approach the right atrium (not shown). The catheter 76 is passed through the interatrial septum (not shown) to reach the left atrium 16. From inside the left atrium 16, the apparatus 10 is then positioned within the annulus 20 of the mitral valve 14 as is shown in FIG. 1. It should be noted that the angular orientation of the apparatus 10 within the mitral valve 14 is important, so radiopaque markers (not shown) may be used to ensure the apparatus is rotated to the proper position prior to deployment.

Next, the catheter 76 is pulled back so that the expandable support member 32 can expand to the condition shown in FIG. 2 in the annulus 20 of the native mitral valve 14. The balloon 74 is then inflated, which pushes the main body portion 44 of the expandable support member 32 into engagement with the annulus 20 as shown in FIG. 3.

The constraining wires are then released, which allows the upper wing members 48 and the lower wing members 56 of the expandable support member 32 to spring radially outward toward their extended conditions illustrated in FIGS. 2-8. The upper wing members 48, in their radially extended condition, extend transverse to the direction of blood flow through the prosthetic valve 12. Simultaneously, the lower wing members 56 move from their radially collapsed condition toward their radially extended condition. In their radially extended condition, the upper and lower wing members 48 and 56 are circumferentially positioned about the superior and inferior aspects 78 and 80 of the mitral valve annulus 20, respectively. The first and second magnetic components 50 and 58 of the upper and lower wing members 48 and 56 (respectively) are magnetically attracted and pull the upper and lower wing members toward each other. The upper and lower wing members 48 and 56 respectively embrace the superior and inferior aspects 78 and 80 of the mitral valve annulus 20 and, consequently, secure the prosthetic valve 12 in the annulus of the native mitral valve 14. With the apparatus 10 fully deployed, the balloon 74 is deflated and moved out of the mitral valve annulus 20.

In an alternative embodiment of the present invention shown in FIG. 11, the first and second magnetic components 50 and 58 may comprise the magnetic members 52 and 54, respectively. The magnetic members 52 and 54 are attached to the wing members 48 and 56, respectively, by the magnetic force between the magnetic member and the metal of the stent 81. Alternatively, the magnetic members 52 and 54 may be attached to the wing members 48 and 56 by gluing, suturing, pinning, clipping, or any other suitable attachment means. As illustrated in FIG. 11, the upper and lower wing members 48 and 56 of the apparatus 10 firmly engage the superior and inferior aspects 78 and 80 (respectively) of the valve annulus 20 as a result of the magnetic force between the first and second magnetic components 52 and 54. Consequently, the prosthetic valve 12 is secured in the annulus 20 of the native mitral valve 14, for example.

It should be noted that the engagement of the main body portion 44 with the valve annulus 20, the engagement of the upper wing members 48 with the wall of the left atrium 16, and the engagement of the lower wing members 56 that pins the native mitral valve leaflets 22 and 24 back against the mitral valve annulus provides a unique three-way locking mechanism for securing the apparatus 10 in the mitral valve annulus.

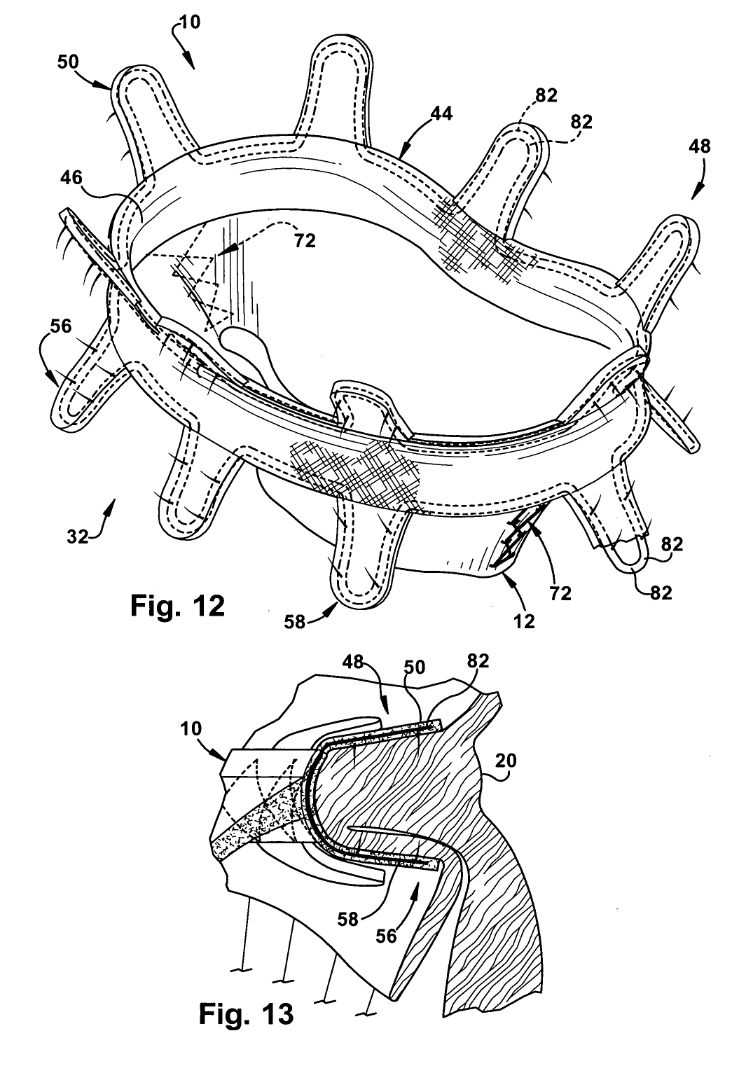

As illustrated in the alternative embodiment of FIGS. 12 and 13, the first and second magnetic components 50 and 58 may also comprise magnetized wires 82. The magnetized wires 82 may be disposed circumferentially about the wing members 48 and 56, and may be comprised of a material capable of producing a magnetic field. Suitable materials include, for example, NdFeB, SmCo, and Alnico. Further, the magnetized wires 82 may be capable of producing a ferromagnetic or non-ferromagnetic field, and may comprise a metal, polymer, ceramic, etc. When the apparatus 10 is in the radially extended condition (FIGS. 2-8), the upper and lower wing members 48 and 56 are pulled toward one another by the magnetic force between the first and second magnetic components 50 and 58 formed by the magnetic wires 82. The upper and lower wing members 48 and 56 respectively embrace the superior and inferior aspects 78 and 80 of the valve annulus 20 and secure the prosthetic valve 12 in the annulus of the native mitral valve 14 as shown in FIG. 13. A benefit of the embodiments illustrated in FIGS. 11-13 is that they allow the thickness of the magnetic components 50 and 58 to be reduced. The reduced thickness serves to make the apparatus 10 easier to load into a catheter for delivery.

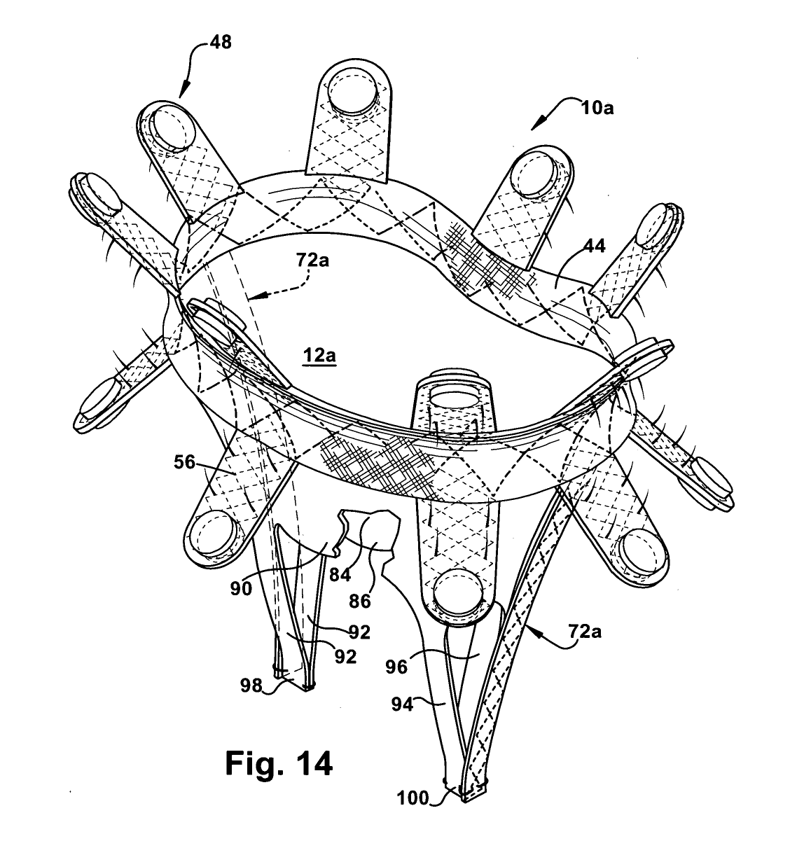

FIG. 14 illustrates an alternative embodiment of the present invention. The apparatus 10ₐ of FIG. 14 is identically constructed as the apparatus 10 of FIGS. 2-8, except whereas described below. In FIG. 14, structures that are identical as structures in FIGS. 2-8 use the same reference numbers, whereas structures that are similar but not identical carry the suffix “a”.

As shown in FIG. 14, the apparatus 10ₐ includes an expandable support member 32 having a flexible configuration and a prosthetic valve 12. The expandable support member 32 is annular in shape and includes oppositely disposed first and second ends 42 and 38 with a main body portion 44 extending between the ends. The apparatus 10ₐ may further include a layer 46 of biocompatible material covering at least a portion of the expandable support member 32.

The first and second ends 42 and 38 of the expandable support member 32 respectively comprise a plurality of upper and lower wing members 48 and 56 that extend integrally from the main body portion 44. The upper and lower wing members 48 and 56 are movable from the radially collapsed condition of FIG. 1 to the radially extended condition of FIG. 14. Each of the upper wing members 48 include a first magnetic component 50, and each of the lower wing members 56 include a second magnetic component 54.

The prosthetic valve 12 of the apparatus 10ₐ may comprise a stentless prosthetic valve, for example, having dimensions that correspond to the dimensions of the native mitral valve 14. Where the prosthetic valve 12 is comprised of biocompatible material, the biocompatible material can include a harvested biological material such as bovine pericardial tissue, equine pericardial tissue, porcine pericardial tissue, animal or human peritoneal tissue, or mitral, aortic, and pulmonary xenograft or homograft. The biocompatible material may also include a suitable synthetic material such as polyurethane, expanded PTFE, woven velour, Dacron®, heparin-coated fabric, or Gor-Tex®.

The prosthetic valve 12 further includes first and second leaflets 84 and 86 that mimic the three-dimensional anatomical shape of the anterior and posterior leaflets 22 and 24, respectively, of the mitral valve 14. The valve leaflets 84 and 86 of the prosthetic valve 12 are joined together at at least two commissural sections 70 that are spaced apart from each other. The prosthetic valve 12 also includes a distal end 88 that defines a first annulus 90 at which the first and second leaflets 84 and 86 terminate.

Additionally, the prosthetic valve 12 includes first and second pairs 92 and 94, respectively, of prosthetic chordae 96 that project from the first and second leaflets 84 and 86 at the first annulus 90. Each of the prosthetic chordae 96 comprises a solid uninterrupted extension of biocompatible material. Each of the first pair 92 of prosthetic chordae 96 has a distal end 98 and each of the second pair 94 of prosthetic chordae has a distal end 100.

As shown in FIG. 14, the second end 38 of the expandable support member 32 may additionally include at least two strut members 72ₐ spaced apart from each other. Each of the at least two commissural sections 70 of the prosthetic valve 12 are attached to a respective one of the strut members 72ₐ to prevent prolapse of the valve leaflets 84 and 86. The strut members 72ₐ are integrally connected to the expandable support member 32 and extend in a generally axial manner along the prosthetic valve 12. The strut members 72ₐ may be attached to the distal ends 98 of the first pair 92 of the prosthetic chordae 96 by sutures, for example. Alternatively, the strut members 72ₐ may be attached to the distal ends 100 of the second pair 94 of the prosthetic chordae 96. It is contemplated that the configuration of the strut members 72ₐ may be varied as needed. For instance, the strut members 72ₐ may have a shorter length than the length of the strut members illustrated in FIG. 14. In this instance, the strut members 72ₐ may be attached at a position proximal to the distal ends 98 and 100 of the prosthetic chordae 96, such as at or near the first annulus 90 of the prosthetic valve 12.

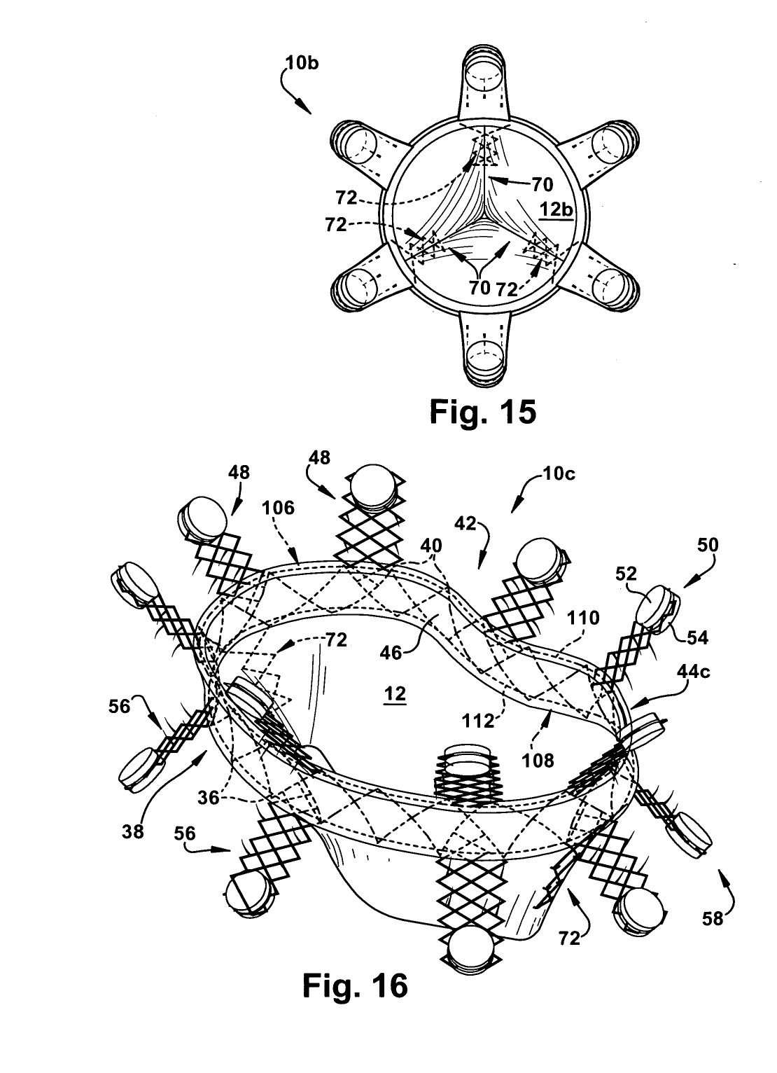

FIG. 15 illustrates another alternative embodiment of the present invention. The apparatus 10_{b} of FIG. 15 is identically constructed as the apparatus 10 of FIGS. 2-8, except whereas described below. In FIG. 15, structures that are identical as structures in FIGS. 2-8 use the same reference numbers, whereas structures that are similar but not identical carry the suffix “b”.

As shown in FIG. 15, the apparatus 10_{b} comprises a tri-leaflet prosthetic valve 12_{b}. The tri-leaflet prosthetic valve 12_{b}, such as a porcine aortic valve, may be used in either the mitral or tricuspid position. The prosthetic valve 12_{b} may be made of other biological materials, including, but not limited to, aortic xenografts, bovine pericardial tissue, equine pericardial tissue, porcine pericardial tissue, peritoneal tissue, and a homograft or allograft. Additionally, the prosthetic valve 12_{b} may be made of any one or combination of biocompatible materials such as polyurethane, PTFE, expanded PTFE, Dacron®, woven velour, Gore-Tex®, and heparin-coated fabric.

As may be seen in FIG. 15, in the tricuspid position, six lower wing members 56 may be used so that a lower wing member is positioned at each commissural section 70 and directly over each native valve leaflet. The expandable support member 32 of the apparatus 10_{b} also includes at least three strut members 72 that are spaced apart from each other. The valve leaflets of the prosthetic valve 12_{b} are joined together at at least three commissural sections 70. Each of the three commissural sections 70 are attached to a representative one of the strut members 72 to prevent prolapse of the valve leaflets. Other than this, the apparatus 10_{b} with the tri-leaflet prosthetic valve 12_{b} is deployed and functions as described above with regard to the previous embodiment. It should be understood that more or less than six lower wing members 56 could be used.

FIG. 16 illustrates another alternative embodiment of the present invention. The apparatus 10c of FIG. 16 is identically constructed as the apparatus 10 of FIGS. 2-8, except whereas described below. In FIG. 16, structures that are identical as structures in FIGS. 2-8 use the same reference numbers, whereas structures that are similar but not identical carry the suffix “c”.

As shown in FIG. 16, the apparatus 10c comprises an expandable support member 32 having oppositely disposed first and second ends 42 and 38 and a main body portion 44_{c} extending between the ends. The first and second ends 42 and 38 of the expandable support member 32 respectively comprise a plurality of upper and lower wing members 48 and 56 that extend from the main body portion 44_{c} and are spaced circumferentially apart about the main body portion. The upper and lower wing member 48 and 56 respectively comprise first and second magnetic components 50 and 58, and may further comprise at least one attachment mechanism 102. The second end 38 of the apparatus 10_{c} also includes at least two strut members 72 that are spaced apart from each other. The apparatus 10 further comprises a prosthetic valve 12 secured within the main body portion 44_{c} of the expandable support member 32. The prosthetic valve 12 has at least two native valve leaflets that are joined together at at least two commissural sections 70 that are spaced apart from each other, and which are attached to a respective one of the strut members 72.

As may be seen in FIG. 16, the main body portion 44_{c} of the expandable support member 32 further comprises a first end portion 106 and a second end portion 108. Securely attached to the first and second end portions 106 and 108 are first and second magnetic ring components 110 and 112. The first and second magnetic ring components 110 and 112 are flexible and may be shaped like a ring or band. The first and second magnetic ring components 110 and 112 may be securely attached to the first and second end portions 106 and 108, respectively, using a suture or adhesive, for example. More particularly, the first magnetic ring component 110 may be attached to the upper tips 40 of the W-shaped segments 34 comprising the first end portion 106, and the second magnetic ring component 112 may be attached to the lower tips 36 of the W-shaped segments comprising the second end portion 108. As shown in FIG. 16, the first and second magnetic ring components 110 and 112 are respectively “threaded” through the upper and lower tips 40 and 36 of the main body portion 44_{c}. Alternatively, the first and second magnetic ring components 110 and 112 may wrap around the exterior or interior surfaces of the first and second end portions 106 and 108, respectively, of the main body portion 44_{c}. The first and second magnetic ring components 110 and 112 are comprised of material capable of producing a magnetic field. Examples of suitable materials include NdFeB, SmCo, and Alnico.

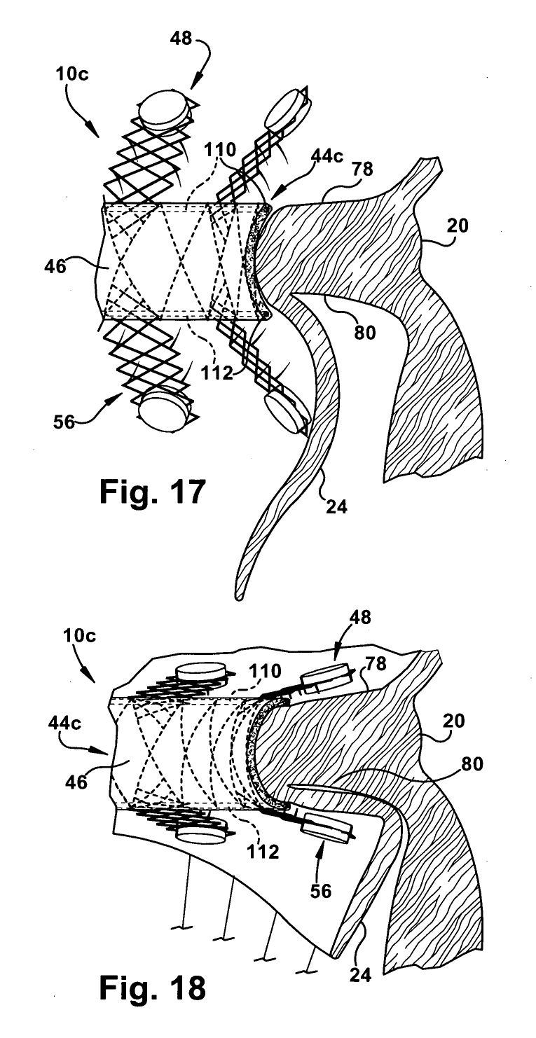

The first and second magnetic ring components 110 and 112 facilitate placement of the expandable support member 32 in the annulus 20 of the mitral valve 14, for example. When the expandable support member 32 is first placed in the mitral annulus 20 as shown in FIG. 17, the first and second magnetic ring components 110 and 112 are oppositely disposed about the superior and inferior aspects 78 and 80 of the annulus, respectively. After placement of the expandable support member 32, the first and second magnetic ring components 110 and 112 are magnetically attracted to one another so that the first and second end portions 106 and 108 of the main body portion 44_{c} are pulled toward one another to secure the expandable support member in the annulus 20 (FIG. 18). Consequently, a tighter seal is formed between the expandable support member 32 and the annulus 20 which, in turn, prevents unwanted blood flow in the space between the expandable support member and the annulus.

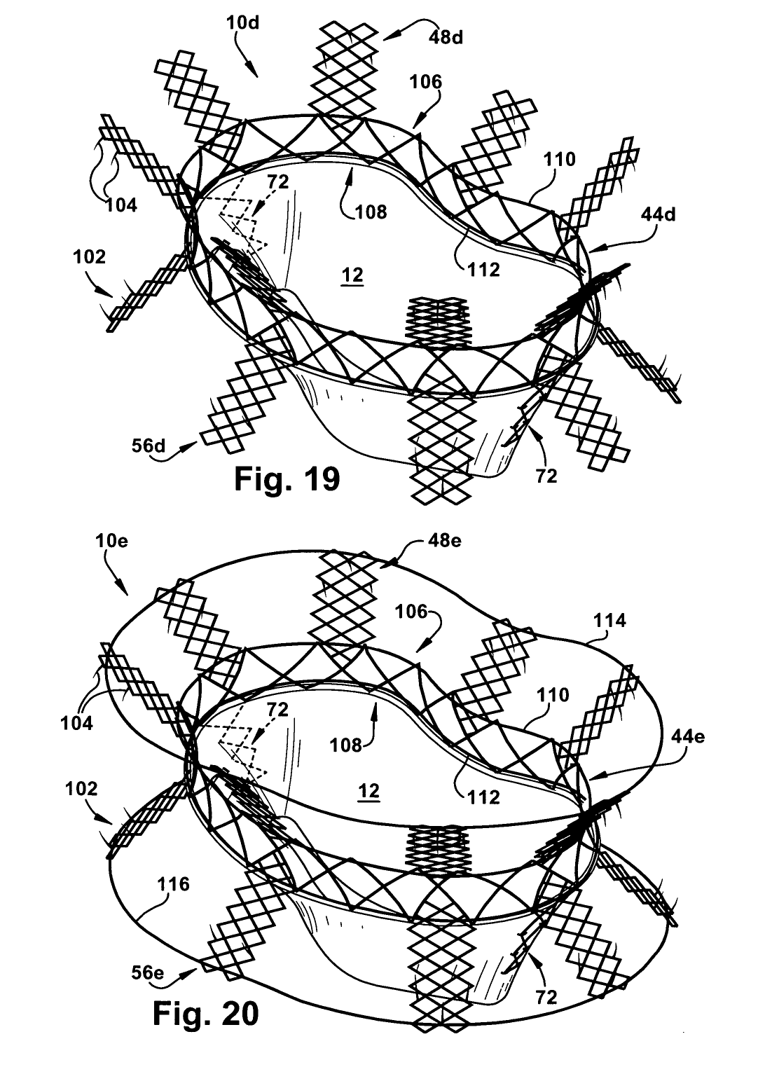

FIG. 19 illustrates yet another alternative embodiment of the present invention. The apparatus 10_{d} of FIG. 19 is identically constructed as the apparatus 10 of FIGS. 2-8, except whereas described below. In FIG. 19, structures that are identical as structures in FIGS. 2-8 use the same reference numbers, whereas structures that are similar but not identical carry the suffix “d”.

As shown in FIG. 19, the apparatus 10_{d} comprises an expandable support member 32 having oppositely disposed first and second ends 42 and 38 and a main body portion 44_{d} extending between the ends. The first and second ends 42 and 38 of the expandable support member 32 respectively comprise a plurality of upper and lower wing members 48_{d} and 56_{d} that extend from the main body portion 44_{d} and are spaced circumferentially apart about the main body portion.

The upper and lower wing member 48_{d} and 56_{d} each include at least one attachment mechanism 102. The attachment mechanism 102 can include at least one barb 104, hook (not shown), or other similar means for embedding into a section of cardiac tissue. For example, where each of the upper wing members 48_{d} include at least one barb 104, the barb or barbs may embed into a section of cardiac tissue to help secure the expandable support member 32 in the annulus 20 of the mitral valve 14. Additionally, where each of the lower wing members 56_{d} include at least one barb 104, the barb or barbs may embed into a portion of the native valve leaflets 22 and 24 to help secure the expandable support member 32 in the annulus 20 of the valve 14.

As shown in FIG. 19, the second end 38 of the apparatus 10_{d} also includes at least two strut members 72 that are spaced apart from each other. Further, the apparatus 10_{d} includes a prosthetic valve 12 secured within the main body portion 44_{d} of the expandable support member 32. The prosthetic valve 12 has at least two native valve leaflets that are joined together at at least two commissural sections 70 that are spaced apart from each other, and which are attached to a respective one of the strut members 72.

The main body portion 44_{d} of the expandable support member 32 further comprises a first end portion 106 and a second end portion 108. Securely attached to the first and second end portions 106 and 108 are first and second magnetic ring components 110 and 112. The first and second magnetic ring components 110 and 112 are flexible and may be shaped like a ring or band. The first and second magnetic ring components 110 and 112 may be securely attached to the first and second end portions 106 and 108, respectively, using a suture or adhesive, for example. More particularly, the first magnetic ring component 110 may be attached to the upper tips 40 of the W-shaped segments 34 comprising the first end portion 106, and the second magnetic ring component 112 may be attached to the lower tips 36 of the W-shaped segments comprising the second end portion 108. As shown in FIG. 19, the first and second magnetic ring components 110 and 112 are respectively “threaded” through the upper and lower tips 40 and 36 of the main body portion 44_{d}. Alternatively, the first and second magnetic ring components 110 and 112 may wrap around the exterior or interior surfaces of the first and second end portions 106 and 108, respectively, of the main body portion 44_{d}. The first and second magnetic ring components 110 and 112 are comprised of material capable of producing a magnetic field. Examples of suitable materials include NdFeB, SmCo, and Alnico.

The first and second magnetic ring components 110 and 112 facilitate placement of the expandable support member 32 in the annulus 20 of the mitral valve 14, for example. When the expandable support member 32 is first placed in the mitral annulus 20, the first and second magnetic ring components 110 and 112 are oppositely disposed about the superior and inferior aspects 78 and 80 of the annulus, respectively. After placement of the expandable support member 32, the first and second magnetic ring components 110 and 112 are magnetically attracted to one another so that the first and second end portions 106 and 108 of the main body portion 44_{d} are pulled toward one another to secure the expandable support member in the annulus 20. Consequently, a tighter seal is formed between the expandable support member 32 and the annulus 20 which, in turn, prevents unwanted blood flow in the space between the expandable support member and the annulus.

FIG. 20 illustrates yet another alternative embodiment of the present invention. The apparatus 10e of FIG. 20 is identically constructed as the apparatus 10_{d} of FIG. 19, except whereas described below. In FIG. 20, structures that are identical as structures in FIG. 19 use the same reference numbers, whereas structures that are similar but not identical carry the suffix “d”.

As shown in FIG. 20, the apparatus 10ₑ comprises an expandable support member 32 having oppositely disposed first and second ends 42 and 38 and a main body portion 44ₑ extending between the ends. The first and second ends 42 and 38 of the expandable support member 32 respectively comprise a plurality of upper and lower wing members 48ₑ and 56ₑ that extend from the main body portion 44ₑ and are spaced circumferentially apart about the main body portion.

As shown in FIG. 20, third and fourth magnetic ring components 114 and 116 are securely attached to the upper and lower wing members 48ₑ and 56ₑ, respectively. The third and fourth magnetic ring components 114 and 116 are flexible and may be shaped like a ring or band. The third and fourth magnetic ring components 114 and 116 may be respectively attached to the upper and lower wing members 48ₑ and 56ₑ using a suture or adhesive, for example. More particularly, the third and fourth magnetic ring components 114 and 116 may be respectively attached to the upper and lower wing members 48ₑ and 56ₑ by “threading” the third and fourth magnetic ring components through the W-shaped segments 34 comprising the upper and lower wing members. Alternatively, the third and fourth magnetic ring components 114 and 116 may wrap around the upper or lower surfaces of the upper and lower wing members 48ₑ and 56ₑ, respectively. The third and fourth magnetic ring components 114 and 116 are comprised of material capable of producing a magnetic field. Examples of suitable materials include NdFeB, SmCo, and Alnico.

The third and fourth magnetic ring components 114 and 116 facilitate placement of the expandable support member 32 in the annulus 20 of the mitral valve 14, for example. When the expandable support member 32 is first placed in the mitral annulus 20, the third and fourth magnetic ring components 114 and 116 are oppositely disposed about the superior and inferior aspects 78 and 80 of the annulus, respectively. After placement of the expandable support member 32, the third and fourth magnetic ring components 114 and 116 are magnetically attracted to one another so that the upper and lower wing members 48ₑ and 56ₑ are pulled toward one another to secure the expandable support member in the annulus 20.

The present invention thus allows for the apparatus 10 to be delivered in a cardiac catheterization laboratory with a percutaneous approach under local anesthesia using fluoroscopic as well as endocardiographic guidance, thereby avoiding general anesthesia and highly invasive open heart surgery techniques. This approach offers tremendous advantages for high risk patients with severe valvular disease. It should be understood, however, that the present invention contemplates various other approaches, including standard open heart surgeries as well as minimally invasive surgical techniques. Alternatively, it is contemplated that the apparatus 10 could be placed by a retrograde, percutaneous approach. For example, the apparatus 10 may be urged in a retrograde fashion through a femoral artery (not shown), across the aortic arch (not shown), through the aortic valve (not shown), and into the left ventricle 18 where the apparatus may then be appropriate positioned in the native mitral valve 14. Because the present invention omits stitching of the apparatus 10 in the valve annulus 20, surgical time is reduced regardless of whether an open or percutaneous approach is used.

From the above description of the invention, those skilled in the art will perceive improvements, changes and modifications. For example, it should be understood by those skilled in the art that the various portions of the expandable support member 32 could be self-expanding or expanded by a change in temperature (because they are made from a shape memory material). Further, it is contemplated that conventional hooks or barbs (not shown) could be used along with the magnetic attachment scheme to provide a redundant or back-up securing mechanism. Such improvements, changes and modifications within the skill of the art are intended to be covered by the appended claims.

Having described the invention, we claim:

1. An apparatus for replacing a cardiac valve having at least two native valve leaflets, said apparatus comprising:
an expandable support member having oppositely disposed first and second ends and a main body portion extending between said ends, said main body portion of said expandable support member having an annular shape for expanding into position in the annulus of the cardiac valve;
said first end of said expandable support member comprising a plurality of upper wing members that extend from said main body portion and are spaced circumferentially apart about said main body portion, each of said upper wing members having a first magnetic component;
said second end of said expandable support member comprising a plurality of lower wing members that extend from said main body portion, each of said lower wing members having a second magnetic component;
said second end of said expandable support member further including at least two strut members that are spaced apart from each other;
said first and second magnetic components being magnetically attracted to one another so that, when said apparatus is placed in the annulus of the cardiac valve, said upper and lower wing members are pulled toward one another to secure said expandable support member in the annulus; and
a prosthetic valve secured within said main body portion of said expandable support member, said prosthetic valve having at least two valve leaflets that are coaptable to permit unidirectional flow of blood, each of said at least two valve leaflets being joined together at at least two commissural sections that are spaced apart from each other, each of said at least two commissural sections being attached to a respective one of said strut members to prevent prolapse of said valve leaflets.

2. The apparatus of claim 1 wherein said main body portion further comprises a first end portion and a second end portion, said first and second end portions respectively having first and second magnetic ring components.

3. The apparatus of claim 1 wherein said plurality of lower wing members includes first and second lower wing members for positioning at the commissures of the cardiac valve.

4. The apparatus of claim 3 wherein said plurality of lower wing members further includes third and fourth lower wing members for positioning directly over respective central portions of the at least two native valve leaflets.

5. The apparatus of claim 1 wherein at least one of said upper and lower wing members each include at least one attachment mechanism for securing said expandable support member in the annulus of the cardiac valve.

6. The apparatus of claim 5 wherein said at least one attachment mechanism includes at least one barb.

7. The apparatus of claim 1 wherein at least a portion of said expandable support member is covered with a layer of biocompatible material.

8. The apparatus of claim 1 wherein at least a portion of said apparatus is comprised of a bioabsorbable material.

9. The apparatus of claim 1 wherein at least a portion of said expandable support member is treated with at least one therapeutic agent for eluting into cardiac tissue or a cardiac chamber.

10. The apparatus of claim 1 wherein a plurality of portions of said expandable support member are separately treated with a different one of said at least one therapeutic agent.

11. The apparatus of claim 10 wherein said main body portion and said wing members are each separately treated with a different one of said at least one therapeutic agent.

12. The apparatus of claim 10 wherein each of said wing members is separately treated with a different one of said at least one therapeutic agent.

13. A method for replacing a cardiac valve having at least two native valve leaflets, said method comprising the steps of:
providing a prosthetic valve that includes an expandable support member having a main body portion, a plurality of upper wing members that extend from a first end of the main body portion and which include a first magnetic component attached to each upper wing member, and a corresponding plurality of lower wing members that extend from an opposite second end of the main body portion and which include a second magnetic component attached to each lower wing member, the second end of the expandable support member further including at least two strut members, the prosthetic valve having at least two valve leaflets that are joined together at at least two commissural sections, each of the at least two commissural sections being attached to a respective one of the strut members to prevent prolapse of the valve leaflets, the prosthetic valve having at least two valve leaflets that are coaptable to permit unidirectional flow of blood;
placing the main body portion of the prosthetic valve within the annulus of the cardiac valve to be replaced;
expanding the main body portion into engagement with the annulus of the cardiac valve to secure the prosthetic valve in the annulus; and
deploying the upper and lower wing members from a radially collapsed condition into a radially extended condition whereby the first and second magnetic components are magnetically attracted and pull the upper and lower wing members toward each other, which secures the prosthetic valve in the annulus of the native cardiac valve.

14. The method of claim 13 wherein said step of placing the main body portion of the prosthetic valve within the annulus of the cardiac valve to be replaced further comprises the steps of:
placing the expandable support member around an inflatable balloon in a secured manner;
inserting the balloon and expandable support member into an atrial chamber;
advancing the balloon until the expandable support member is positioned within the annulus of the cardiac valve to be replaced; and
expanding the expandable support member with the balloon so that the expandable support member engages the annulus of the cardiac valve to secure the expandable support member in the annulus.

15. The method of claim 14 wherein said step of inserting the balloon and the expandable support member into the atrial chamber is done percutaneously via an intravascular catheter.

16. The method of claim 14 wherein said step of inserting the balloon and the expandable support member into the atrial chamber is done via a minimally invasive, transthoracic approach via a port on the heart wall.

17. The method of claim 14 wherein said step of inserting the balloon and the expandable support member into the atrial chamber is done via an open-chest procedure under direct supervision.

18. The method of claim 13 wherein at least a portion of the expandable support member is treated with at least one therapeutic agent for eluting into cardiac tissue or a cardiac chamber, said method further comprising the step of allowing the at least one therapeutic agent to elute into the cardiac tissue or the cardiac chamber.

19. The method of claim 13 wherein a plurality of portions of the expandable support member are separately treated with a different one of the at least one therapeutic agent.

20. The method of claim 19 wherein the main body portion and the wing members are each separately treated with a different one of the at least one therapeutic agent.

21. The method of claim 19 wherein each of the wing members is separately treated with a different one of the at least one therapeutic agent.

22. The method of claim 13 wherein at least one of the upper and lower wing members each include at least one attachment mechanism for securing the expandable support member in the annulus of the cardiac valve.

23. The method of claim 13 wherein the main body portion further comprises a first end portion and a second end portion, the first and second end portions respectively having first and second magnetic ring components.

24. The method of claim 13 wherein the upper and lower wing members respectively include third and fourth magnetic ring components.

25. An apparatus for replacing a cardiac valve having at least two native valve leaflets, said apparatus comprising:
an expandable support member having oppositely disposed first and second ends and a main body portion extending between said ends, said main body portion of said expandable support member having an annular shape for expanding into position in the annulus of the cardiac valve, said first end of said expandable support member comprising a plurality of upper wing members that extend from said main body portion, said second end of said expandable support member comprising a plurality of lower wing members that extend from said main body portion, said second end of said expandable support member further including at least two strut members that are spaced apart from each other;
said upper and lower wing members including means for magnetically attracting upper and lower wing members toward each other to secure said apparatus in the annulus of the native cardiac valve; and
a prosthetic valve secured within said main body portion of said expandable support member, said prosthetic valve having at least two valve leaflets that are coaptable to permit unidirectional flow of blood, each of said at least two valve leaflets being joined together at at least two commissural sections that are spaced apart from each other, each of said at least two commissural sections being attached to a respective one of said strut members to prevent prolapse of said valve leaflets.

26. The apparatus of claim 25 wherein said means for magnetically attracting upper and lower wing members includes at least one magnetized wire.

27. The apparatus of claim 25 wherein said plurality of lower wing members includes first and second lower wing members for positioning at the commissures of the cardiac valve.

28. The apparatus of claim 27 wherein said plurality of lower wing members further includes third and fourth lower wing members for positioning directly over respective central portions of the at least two native valve leaflets.

29. The apparatus of claim 25 wherein at least a portion of said expandable support member is covered with a layer of biocompatible material.

30. The apparatus of claim 25 wherein at least a portion of said expandable support member is treated with at least one therapeutic agent for eluting into cardiac tissue or a cardiac chamber.

31. The apparatus of claim 25 wherein a plurality of portions of said expandable support member are separately treated with a different one of said at least one therapeutic agent.

32. The apparatus of claim 31 wherein said main body portion and said wing members are each separately treated with a different one of said at least one therapeutic agent.

33. The apparatus of claim 31 wherein each of said wing members is separately treated with a different one of said at least one therapeutic agent.

34. The apparatus of claim 25 wherein at least one of said upper and lower wing members each include at least one attachment mechanism for securing said expandable support member in the annulus of the cardiac valve.

35. An apparatus for replacing a cardiac valve having at least two native valve leaflets, said apparatus comprising:
an expandable support member having oppositely disposed first and second ends and a main body portion extending between said ends, said main body portion having an annular shape for expanding into position in the annulus of the cardiac valve;
said first and second ends of said expandable support member respectively comprising a plurality of upper and lower wing members that extend from said main body portion and are spaced circumferentially apart about said main body portion, each of said upper and lower wing members having at least one attachment mechanism;
said second end of said expandable support member further including at least two strut members that are spaced apart from each other;
said main body portion comprising a first end portion and a second end portion, said first and second end portions respectively having first and second magnetic ring components;
said first and second magnetic ring components being magnetically attracted to one another so that, when said apparatus is placed in the annulus of the cardiac valve, said first and second end portions of said main body portion are pulled toward one another to secure said expandable support member in the annulus; and
a prosthetic valve secured within said main body portion of said expandable support member, said prosthetic valve having at least two valve leaflets that are coaptable to permit unidirectional flow of blood, each of said at least two valve leaflets being joined together at at least two commissural sections that are spaced apart from each other, each of said at least two commissural sections being attached to a respective one of said strut members to prevent prolapse of said valve leaflets.

36. The apparatus of claim 35 wherein said upper and lower wing members respectively include third and fourth magnetic ring components, said third and fourth magnetic ring components being magnetically attracted to one another so that, when said apparatus is placed in the annulus of the cardiac valve, said third and fourth magnetic ring components are pulled toward one another to secure said expandable support member in the annulus.

37. The apparatus of claim 35 wherein said plurality of lower wing members includes first and second lower wing members for positioning at the commissures of the cardiac valve.

38. The apparatus of claim 37 wherein said plurality of lower wing members further includes third and fourth lower wing members for positioning directly over respective central portions of the at least two native valve leaflets.

39. The apparatus of claim 35 wherein said at least one attachment mechanism of said upper wing members is for embedding into a section of cardiac tissue to help secure said expandable support member in the annulus of the cardiac valve.

40. The apparatus of claim 35 wherein said at least one attachment mechanism of said lower wing members is for embedding into a portion of the native valve leaflets to help secure said expandable support member in the annulus of the cardiac valve.

41. The apparatus of claim 35 wherein at least a portion of said expandable support member is covered with a layer of biocompatible material.

42. The apparatus of claim 35 wherein at least a portion of said expandable support member is treated with at least one therapeutic agent for eluting into cardiac tissue or a cardiac chamber.

43. The apparatus of claim 35 wherein a plurality of portions of said expandable support member are separately treated with a different one of said at least one therapeutic agent.

44. The apparatus of claim 43 wherein said main body portion and said wing members are each separately treated with a different one of said at least one therapeutic agent.

45. The apparatus of claim 43 wherein each of said wing members is separately treated with a different one of said at least one therapeutic agent.
